(19) United States
(12) Patent Application Publication
(10) Pub. No.: US 2006/0106405 A1
(43) Pub. Date: May 18, 2006

(54) Systems and methods for delivering fastener to opposed tissue structures

(76) Inventors: James I. Fann, Portola Valley, CA (US); Hanson S. Gifford, Woodside, CA (US)

Correspondence Address: BELL & ASSOCIATES, 416 FUNSTON ST., SUITE 100, SAN FRANCISCO, CA, 94118, US

(21) Appl. No.: 11/274,791

(22) Filed: Nov. 15, 2005

Related U.S. Application Data
(60) Provisional application No. 60/628,745, filed on Nov. 16, 2004.

(51) Int. Cl. A61B 17/10

(52) U.S. Cl. 606/142

(57) ABSTRACT

Flexible clips which are transported over a solid needle or through a hollow needle and methods of use thereof are described for tissue approximation and attachment and for joining a graft vessel to a target vessel. The tissue approximation or anastomosis clip includes a highly flexible or elastic material wrapped around and transported on the outside of a solid needle. Another embodiment is a flexible or composite clip which is transported within a hollow needle. The distal end of the clips has a tapered configuration to minimize resistance and facilitate tissue penetration. After placement of the clip in the desired location, the solid or hollow needle is withdrawn resulting in coiling or contraction of the clip thus approximating the tissue and securing the clip. Multiple clips can be loaded in the deployment device which is calibrated to deploy and secure one clip at a time. An additional embodiment is a needle point attached to a clip that overrides a solid shaft. Deployment is by pushing or displacing the clip portion from the shaft. A plurality of these needle-clips may be oriented on a circular ring which permits rapid clip deployment and tissue approximation, such as for anastomosis, and methods for subsequent ring removal or dismantling.

The present application claims priority to U.S. Provisional Patent Application Ser. No. 60/628,745 entitled “Systems And Methods For Delivering Fastener To Opposed Tissue Structures”, filed Nov. 16, 2004, which is herein incorporated by reference in its entirety for all purposes.

### BACKGROUND OF THE INVENTION

1. Field of the Invention

The present invention relates to surgical devices and methods for tissue approximation and attachment, and anastomosis of hollow organs or blood vessels, particularly coronary artery anastomosis and gastrointestinal anastomosis.

Surgical anastomosis is the connection of two vascular structures or hollow organs, such as a graft vessel and a coronary artery or segments of intestine to each other. In coronary artery disease, the flow of oxygenated blood through the coronary arteries is compromised thereby leading to myocardial ischemia and infarction. Surgical treatment of severe coronary artery disease is coronary artery bypass grafting, in which a graft vessel, such as internal mammary artery, saphenous vein, or radial artery, is anastomosed to the target coronary artery beyond the blockage.

In coronary artery bypass grafting, the technical aspect of the anastomosis of a graft vessel to a coronary artery is challenging because of the small size of the vessels, which are usually 1-3 mm in diameter, and the need to perform this procedure expeditiously in patients on cardiopulmonary bypass for on-pump procedures or during a period of ischemia or potential hemodynamic compromise in off-pump surgery. While on cardiopulmonary bypass or on-pump surgery, it is ideal to minimize the time of cardioplegic arrest and myocardial injury. For off-pump coronary artery bypass grafting, exposure of the target coronary vessel often requires manipulation of the heart and temporary occlusion of the target artery, a process that may result in hemodynamic or electrical instability of the heart. Notwithstanding the use of stabilizing devices for off-pump surgery, motion of the target vessels may pose a further challenge to the surgeon.

The conventional approach to coronary or vascular anastomosis is using a needle with a suture placed either in a continuous or interrupted fashion. After creating a small hole in the target vessel beyond the blockage, the graft vessel is anastomosed to the target artery. The needle typically punctures from the outside to the inside of the graft and from the inside to the outside of the target artery. In diseased target vessels, puncturing the needle from the inside out, i.e., intima to adventitia, decreases the chance of separating the layers in the wall of the blood vessel. Because of the variability in the size and quality of the graft and target vessels, the anastomosis is customized by the surgeon to minimize any anatomic discrepancies. Thus, rigid coupling or anastomotic devices may be difficult to use in situations where there are substantial anatomic discrepancies, and devices that penetrate the target artery from the outside, or adventitia, to the inside, or intima, may result in undue trauma to the vessel.

Recent approaches to minimally invasive coronary artery bypass grafting, such as using smaller incisions with the assistance of endoscopic and robotic technology, require that the vascular anastomosis be performed in a limited field with limited access. In spite of various devices intended to facilitate limited access surgery, the current approach for the graft to target coronary artery anastomosis continues to be based on conventional suturing techniques. In this restricted environment, tasks that are potentially time-consuming include suture tying and suture management. Therefore, anastomosis devices and methods that permit adjustments for anatomic variability, a hemostatic seal, use in a limited access environment and rapid deployment can be of benefit to the surgeon. In gastrointestinal surgery, diseased portion of the stomach or bowel is removed, and the remaining proximal and distal portions are connected in a gastrointestinal anastomosis. In laparoscopic surgery, gastrointestinal anastomosis requires accurate suture placement and suture management which is often challenging and time consuming. Currently, after resection of a segment of the intestine, the proximal and distal remaining ends are brought together using sutures or in some cases stapling devices. A clip that facilitates tissue approximation in a limited field with limited access, such as in endoscopic or laparoscopic surgery, may permit rapid and accurate anastomosis of the gastrointestinal system or of other hollow organs.

Devices and methods that facilitate tissue apposition or performance of cardiovascular or gastrointestinal anastomosis using clips, staples, and anchoring devices have been described.

For example, in U.S. Pat. No. 6,461,365 to Bolduc et al., surgical clips and methods of delivery that facilitate tissue approximation are described. Various configurations of clips, including rigid and flexible designs, permit passage through tissue, and the clips assume a shape that results in tissue apposition. In U.S. Pat. No. 6,254,615 to Bolduc et al., a surgical clip comprising of a clip body and a needle portion extending from the clip body is described. Additionally, there is a retainer disposed outside of the graft and target vessel for retaining the graft and target vessel on the needle portion. The retainer is configured to compress the graft vessel to the target vessel wall. In U.S. Pat. No. 5,976,159 to Bolduc et al., the anastomosis clip includes a clip body having a distal extremity with a distal end and a proximal extremity with a proximal end. The distal end is configured to penetrate through the graft vessel wall near the free end and through the target vessel wall near the opening such that both the distal and proximal ends of the clip body are outside the graft and target vessels. At least a portion of the clip body is shapeable so as to compress the graft vessel wall against the target vessel wall with target vessel lumen in communication with the graft vessel lumen. In U.S. Pat. No. 6,641,593 to Schaller et al., a tissue connector assembly comprising a clip movable between an open configuration and a closed configuration and a mechanical restraining device attached to the clip for restraining the clip in its open configuration is described. A needle may be releasably attached to the clip. The method includes inserting a clip through tissue with the clip being biased in an open position by a restraining device secured to the clip, and removing the restraining device from the clip. In U.S. Pat. No. 6,607,541 to Gardiner et al., a tissue connector assembly having a flexible member and a surgical clip releasably coupled to a flexible member is described. A needle may be secured to one end portion of the flexible member with the surgical clip coupled to the other end portion of the flexible member. A locking device may be used to couple the flexible member to the surgical clip. Also described is a method for drawing tissue portions together with a clip assembly and securing the tissue portions together with the clip assembly. In U.S. Pat. No. 6,551,332 to Nguyen et al., an assembly is described, including a surgical fastener comprising a clip movable between an open configuration and a closed configuration and a biasing member contacting the clip and biasing the clip to its open configuration when the biasing member is actuated. The biasing member and clip both tend to assume the closed configuration when no external forces are applied to them. A needle may be releasably attached to the clip. Although the above described methods and delivery systems permit variability of location of clip placement, minimize anatomic discrepancies, and eliminate the issues associated with compliance with rigid ring-type devices, there is no obvious method to provide for simple and rapid re-loading of the clips. For some of the designs, a shaping mechanism extrinsic to the clip may be required. Importantly, because rapid re-loading and deployment of the clips would require a complex multi-fire assembly, any advantage of the above devices and methods as described compared to conventional suturing is mitigated.

In the following group of patents, the approach to tissue apposition is via the transport of fasteners or clips through a tubular structure. In U.S. Pat. No. 6,113,611 to Allen et al., a surgical fastener made from a shape memory alloy is provided which can access and join internal tissue/material through a small surgical access port or incision. After the fastener is deployed, it assumes a shape that automatically applies to the layers of tissue an appropriate hemostatic compression. The device does not involve an anvil and is intended to achieve compression sufficient for hemostasis. In U.S. Pat. No. 6,447,524 to Knodel et al, a surgical fastener is described for attaching a prosthesis to body tissue formed from a generally planar continuous body member, preferably from elastic material. The device contains resilient barbs and/or legs which project in different directions when deployed. The advantage of this system is that it allows for multiple devices to be placed within the lumen of the deployment instrument. In U.S. Pat. No. 6,562,051 to Bolduc et al, a helical fastener is provided with the first end for enhancing tissue penetration and a second end comprising a coil for receiving longitudinal and rotational forces. The helical fasteners are attached to body tissue by a fastener applicator having a proximal portion comprising a handle and an actuator and an elongate distal portion for housing a plurality of fasteners. A transferring action of the actuator provides longitudinal and rotational movement of the fasteners out of the distal portion and into body tissue. In U.S. Pat. No. 6,663,633 to Peirson, a system for fixation of soft tissue tear including a flexible helical fixation element biased to a predetermined pitch. A hollow, generally helical insertion element is dimensioned to admit at least a distal portion of the fixation element into a lumen thereof. The insertion element is insertable in a screwing motion across the soft tissue tear and is positionable with the central portion bridging the tear. In Pat. Appl. No. 2003/0225420 A1 to Wardle, surgical coils for marking, anchoring, stapling and suturing can be implanted in the body by deforming it to a small cross section profile and then sliding it through a low profile delivery device than deploying from an embodiment of a delivery device at a targeted site. Embodiments of surgical coils when deployed revert back to a coiled configuration and circle tissue at the target site. The system permits placement of multiple coils within a deployment instrument. In U.S. Pat. No. 5,997,556, to Tanner, a device is disclosed, consisting of a delivery system and clips to hold grafts in place inside arteries. These clips consist of linear coils such as form the tips of guidewires, preshaped to a tight secondary coil. These are then deployed through the lumen of the delivery system.

With regard to cardiovascular anastomosis, one disadvantage of the devices and delivery systems described above is that the instrument used for deployment may not permit easy passage of the tubular needle from outside to inside and then back to outside of the vessel in order to leave a minimal amount of foreign material within the lumen of the vessel. But the key problem with the above devices and methods is that when reduced to a size appropriate for coronary artery anastomosis, where vessels of 1 to 3 mm are being joined, the concepts described may no longer be practicable. Another problem is that they all describe the deformation of a solid wire, typically a nitinol wire, which is subject to the superelastic strain limits of this material. For a given diameter of nitinol wire, when the wire relaxes from a straightened configuration (inside a delivery needle) to a coiled configuration (inside the tissue), the coil will have a diameter of approximately ten times the diameter of the wire. Therefore, a 0.004″ diameter wire will form a coil with a minimum diameter of approximately 0.040″, or 1 mm. Such coils are likely to be too large and awkward for use in joining two vessels with a diameter of one or two millimeters and are not likely to be hemostatic.

In. U.S. Pat. No. 4,586,503 to Kirsch, a device is disclosed that holds multiple individual clips for creating a vascular anastomoses. One requirement is that the vessel edges need to be everted outwardly so that a clip can be placed over the tissue edges. The clip is then crimped to deform the leg components of the clip in an inward position and thus approximate the tissue without puncturing the tissue. This device eliminates the compliance issues with rigid ring-type devices and allows one to compensate for vessel size discrepancies; however, it may be difficult to achieve tissue eversion on the small scale of coronary arteries. Additionally, two pairs of forceps may be needed to hold the tissue edges in approximation while a third hand applies the clip, in contrast to conventional suturing. Manipulation of the device and clips as in endoscopic surgery, where access, visualization, and maneuverability of instruments are limited, may be particularly difficult.

In U.S. Pat. No. 5,234,447 to Kaster et al., a device is disclosed consisting of a rigid ring with a plurality of pointed legs extending from the ring axially in the distal direction and a plurality of angled legs extending axially from the ring in the proximal direction. The graft vessel is placed through the middle of the ring and the end is everted over the pointed legs, which puncture the vessel wall and retain it on the ring. The pointed legs are then bent outwardly, and the everted end of the graft vessel and the outwardly-oriented pointed legs are inserted through an arteriotomy in the target vessel so that the pointed legs engage the interior wall of the target vessel. The angled legs on the proximal end of the ring are then bent toward the target vessel to penetrate the outer wall thereof. Although this device has a simple one-piece design and avoids the need to evert the wall of the target vessel over the device, the device maintains a rigid ring structure which results in compliance issues at the anastomosis.

Devices and methods are thus needed to facilitate vascular and coronary artery anastomosis but eliminate the various disadvantages of prior devices. The devices and methods should allow the surgeon to select the ideal locations on the graft and target vessels where the device is to be applied, similar to selecting the location of each stitch in a conventional sutured anastomosis. The devices and methods should be relatively simple to utilize, even at the small scale of the coronary arteries. The devices and methods should be useful for performing end-to-side, end-to-end and side-to-side anastomoses. Furthermore, the devices and methods should produce an anastomosis which is reliably hemostatic and patent, with a degree of compliance comparable to sutured anastomosis. The device system is equally applicable to gastrointestinal anastomosis, particularly in limited access or endoscopic surgery.

2. Description of the Background Art

Pertinent references include U.S. Pat. No. 4,586,503 filed May 1986, Kirsch; U.S. Pat. No. 5,234,447 filed August 1993, Kaster et al.; U.S. Pat. No. 5,830,221 filed November 1998, Stein et al.; U.S. Pat. No. 5,976,159 filed November 1999, Bolduc et al.; U.S. Pat. No. 5,997,556 filed December 1999, Tanner; U.S. Pat. No. 6,113,611 filed October 2000, Allen et al.; U.S. Pat. No. 6,149,658 filed November 2000, Gardiner et al.; U.S. Pat. No. 6,254,615 filed July 2001, Bolduc et al.; U.S. Pat. No. 6,447,524 B1 filed September 2002, Knodel et al.; U.S. Pat. No. 6,461,365 filed October 2002, Bolduc et al.; U.S. Pat. No. 6,562,051 B1 filed May 2003, Bolduc et al.; U.S. Pat. No. 6,607,541 filed August 2003, Gardiner et al.; U.S. Pat. No. 6,641,593 filed November 2003, Schaller et al.; U.S. Pat. No. 6,551,332 filed April 2003, Nguyen et al.; and U.S. Pat. No. 6,663,633 filed December 2003, Peirson, III; and Publication No. 2003/025420 A1 filed December 2003, Wardle.

### BRIEF SUMMARY OF THE INVENTION

The invention provides fasteners such as clips and methods that meet at least some of the foregoing needs and that are useful not only for vascular or coronary artery anastomosis, but for anastomosis of a variety of other hollow organs, such as intestines, as well as in wound closure and other tissue approximation and attachment applications. The invention offers a convenient solution to cardiovascular anastomosis, allowing the anastomosis to be performed with the hemostasis, patency, compliance and reliability of interrupted sutures. The devices and methods of the invention are useful not only in conventional open surgical procedures, but in endoscopic, laparoscopic, thoracoscopic, robotically-assisted, and other minimally-invasive procedures as well.

In one embodiment, the fastener such as a clip is a thin flexible coil or ‘tube’ which travels on the outside of a delivery shaft such as a solid needle. The leading edge of the clip may be tapered flush to the needle thereby decreasing the resistance as it travels through the vessel layers. After tissue penetration the clip is advanced and the needle retracted resulting in clip deployment. The clip may assume a variety of configurations once deployed. The clip may coil onto itself thereby approximating the tissue layers. The clip also may contract and become dumbbell in shape thereby anchoring itself on the outer layers of the tissues. Finally, the clip may assume a V-shape or U-shape which permits it to approximate and compress the tissue layers. In this manner, the clip reproduces the current conventional suture technique and minimizes foreign body to blood contact and the potential complications associated with a rigid or less compliant system. It further eliminates the need for an internal anvil which may be traumatic upon its removal and obviates knot tying. Because of the inert and the elastic quality of the material, the clip provides for reliable and durable connection. Moreover, through the use of a plurality of individual surgical clips, the clip provides the surgeon with the flexibility to select the optimal location of clip placement on both the target and graft vessel walls.

This tubular clip could be made from a simple wire coil. For instance, a clip made for coronary artery anastomosis might pass over a needle of approximately 0.007″ in diameter, and the coil might have an inner diameter of 0.0075″ and an outer diameter of 0.0095″. The wire coil in this example might be wound from a single wire with a diameter of 0.001″, but it might also be wound from a number of wires, for example four wires, which would make it much stronger. The clip might be made from many more wires, until the wires might run much more longitudinally, and less circumferentially, around the delivery needle. The wires might also have a square cross-section, rather than a round cross-section. These might be bare wires, or they might again be held together with an adhesive or polymer coating of appropriate materials as described above.

One significant advantage of these tubular clips is that they might be formed with ‘heads’ on their proximal ends. These heads might be formed from the wires, or they might be a separate element, or they might be a continuation of the polymer on the clip, or they might be made using other constructions. In use, the delivery needle and the distal portion of the tubular clip would be passed through the tissue until the tissue pressed against the head of the clip, which would be large enough not to pass through the tissue. While holding enough forward pressure on the clip to prevent the tissue from sliding distally, the needle would be retracted, allowing the distal portion of the clip to reform into a coil. The tissue would remain compressed between the proximal head and the distal coil. The head on the clip would not only simplify the surgical issues associated with positioning and delivering the clips appropriately, it would also reduce the volume of material associated with one side of the clip to the absolute minimum needed to prevent it from passing through the tissue.

Another embodiment comprises a flexible composite clip that travels within the lumen of a hollow needle. The hollow needle may be completely cylindrical or partially cylindrical. The latter design permits a lower profile for the needle and a relatively larger clip. The tip of the clip lies flush with the tip of the needle. After penetration of the tissue layers, the clip is advanced as the needle is retracted. With needle retraction, the clip assumes the shape that is pre-determined, thereby compressing and approximating the tissue layers. While the clip may be a single solid wire of NITINOL or other highly elastic material, the clip is likely to be more complex in construction. For instance, it may be made of a number of wires, for example seven wires, in order to dramatically reduce the coil diameter when the clip is deployed. Whereas a solid wire of 0.004″ would have a deployed coil diameter of approximately 0.040″, a wire made of seven wires, each with a diameter of 0.0013″, could relax into a coil with a diameter of approximately 0.013″. This would be a significant advantage for anastomosis of very small vessels. These seven smaller wires might be twisted to hold them together, both when straight and when released into a coil shape. Another, alternative construction would be to take seven wires with a diameter of 0.001″, and to surround them in a tightly wound coil of wire (or ribbon) with a diameter (or thickness) of 0.0005″, again forming a ‘wire’ with an overall diameter of 0.004″. The ‘wire’ might also have a composite construction, with a number of NITINOL or stainless steel wires held together by an adhesive or polymer. It might be advantageous for this polymer to allow the wires to slide relative to each other, to preserve their ability to relax into a smaller diameter coil. The number of smaller wires might, for instance, be held together within a thin-walled tube of a polymer such as polytetrafluorethylene (PTFE), ePTFE, polypropylene, or another polymer. Another alternative construction would be for the clip to be formed from one or more coils of one or more wires. For example, the clip might be formed from two concentric coils, each made from four individual strands of wire. Yet another alternative construction would be for the clip to be formed from a stack of thin ribbons of elastic material, again possibly held together by a polymer coating or casing. By using thin ribbons, each ribbon could again be pre-formed to reform into a much tighter coil than a single thick wire.

The previous paragraph describes how the clip can be designed to form a small diameter coil by making it from several smaller wires or ribbons. However, the clip may be designed to reform into shapes other than a coil. For instance, the smaller wires might reform into a floret pattern, or another flat shape, or even a random pattern resembling a microscopic ‘BRILLO’ pad, all while still resulting in a smaller final shape or profile due to the smaller diameter bends in each individual wire element. For the partially cylindrical needle, the roof of the needle is cut away to provide a “rail” on which the clip travels. Using this needle design, the clip may assume a larger profile since a smaller portion would actually be in the needle.

The invention also provides a device for clip application or deployment. One embodiment might be used for the device with the tubular clips advancing over the outside of a solid needle. The solid needle or other shaft could extend or be extended proximally along the device, and a number of clips could be serially pre-loaded onto the needle. As each clip is deployed into the tissue, the next clip could be advanced into position near the distal tip of the needle. In the second embodiment, the needle or other shaft is hollow with a taper tip, which permits it to be flush with the tip of the clip in order to decrease resistance during passage of the clip. The needle is either curved, e.g., semi-circular, like conventional suture needles, or straight, or another curve as appropriate to the surgery being performed. The hollow needle is either attached to a hollow tube or itself continues as a hollow tube through which a number of clips can be lined serially.

At the end of the line of clips, there is usually a pusher system which permits clip advancement during needle retraction at time of anastomosis. The pusher mechanism is calibrated to permit advancement of one clip at a time. In order to make certain that the appropriate portion of the clip is deployed on each side of the tissue, actuation of the delivery system might advance approximately half of the clip out of the needle; withdrawing the needle from the tissue would cause the other half of the clip to deploy on the other side of the needle. Alternatively, a multi-pronged “transport” system can be used to deliver the clip and advance subsequent clips. The delivery system could also incorporate an outer ‘stop’ over the needle. The needle would be advanced through the tissue until the tissue presses against this stop. The stop then holds the tissue in the appropriate position as mechanism advances the clip and/or retracts the needle to automatically leave the clip in the right position relative to the tissue. This will simplify the use of this device for the surgeon.

Additionally, the deployment device with the needle is configured to permit endoscopic, thoracoscopic, laparoscopic or robotically assisted approaches by providing a low profile shaft which will enable it to be passed through a trocar into the body cavity of interest. An actuator at the proximal end of the shaft permits remote application of the clip.

Another feature of the clip design is the ability to approximate tissues and perform an anastomosis with a plurality of clips mounted on a ring, which can be dismantled or fragmented. In this embodiment, the clip portion is incorporated with a solid needle tip. This configuration permits multiple needle-clips to be passed through a graft, for example, followed by the rapid serial placement through the target artery. The ring is retracted and the needle-clips are deployed simultaneously. The clip portion of the needle-clip assumes a coil or dumbbell shape thereby approximating the tissues and achieving an anastomosis. The ring can then be removed by dismantling or fragmentation. In order to maintain needle orientation after preloading on the graft and prior to placing the needles through the target vessel, the coil portion rides on a non-cylindrical shaft, such as a shaft that is keyed or square in cross section. Another embodiment of this needle-clip on a ring concept is for the clip component to be traveling within hollow tubes which are mounted in a circular fashion. Again, after tissue penetration, the ring with hollow tubes may be withdrawn with resultant clip assuming either a coil or dumbbell shape for tissue approximation. Because multiple clips can be delivered this fashion, a rapid anastomosis can be achieved.

The invention also provides for a mechanism for clip removal. The clip is designed to permit simple disengagement if misdeployed. Using a pair of forceps, the clip can be removed from the tissue layers. Should this not be possible, a straightening tool will facilitate removal of the clip.

The following drawings and detailed descriptions are intended to illustrate the features and advantages of the invention.

### BRIEF DESCRIPTION OF THE DRAWINGS

FIG. 1. An elastic or highly flexible tubular clip, which is in a coiled configuration, travels over a solid needle with a taper tip. The leading edge of the clip is tapered so as to be flush with the solid needle tip in order to minimize resistance when passing through tissue or vessel layers. The clips can be loaded in series and each subsequent clip can be advanced after each clip deployment. 12=curve of the needle; 14=tip of needle; 10=the clip; 11=the taper on the clip.

FIG. 2. The tubular clip may be constructed of a thin flexible coil or a polymer ‘tube’ which travels on the outside of a solid needle. After tissue penetration the clip is advanced and the needle retracted resulting in clip deployment. The wire coil may be wound from a single wire, or it may be wound from multiple wires, in which case the clip would be stronger. 15=multifilaments of the clip.

FIG. 3. The elastic tubular clip may assume a variety of configurations once deployed. The clip may assume a coiled or contracted configuration once released thereby becoming a tissue clip. The clip may also become V-shape or U-shape which permits it to approximate and compress the tissue layers. The clip may assume dumbbell in shape thereby anchoring itself on the outer layers of the tissues.

FIG. 4. The tubular clips may be formed with ‘heads’ or ‘stops’ on their proximal ends (away from the tip of the clip). These heads may be formed from the wires, or they may be a separate element, or they may be a continuation of the polymer on the clip. During deployment, the clip is advanced until the head, which is large enough not to pass through the tissue, is in contact with the tissue. The needle is retracted and the clip released, thereby assuming a coiled or U-shape configuration. 16=the stop at the end of the clip.

FIG. 5. The delivery needle and the distal portion of the tubular clip would be passed through the layers of the tissues. The needle is retracted, allowing the clip to reform into a coil or other configuration, such as V-shape or U-shape. The tissue would remain compressed between the proximal and the distal portions of the coil. 17=‘U’ shape of the clip.

FIG. 6. The clip may be slotted or “keyed” with a square cross section, for instance. The solid needle over which the clip travels may also be slotted or have a square cross-section, rather than a round cross-section, in order to maintain clip orientation. 19=the keyed slot for the solid needle; 40=the slot on the clip; 18=the square configuration inside the clip; 41=the square configuration for the solid needle over which 18 rides.

FIG. 7. The invention also provides a device for clip application or deployment. This tubular clip is placed in series over the needle to permit rapid multiple firings. With each activation, the clip is advanced and deployed, followed by a subsequent clip which is ready for deployment. The needle and clips are connected to a handle with a pusher or other actuating mechanism which permits calibrated passage and advancement of each individual clip. The solid needle could extend or be extended proximally along the device, and a number of clips could be serially pre-loaded onto the needle and cartridge. As each clip is deployed into the tissue, the next clip could be advanced into position near the distal tip of the needle. 21=the housing for the clip-needle; 26=the push rod to push the clips; 20=the actuating mechanism for moving the clips over the needle.

FIG. 8. A flexible composite clip may travel within the lumen of a hollow needle. The tip of the clip is sharp and lies flush with the tip of the needle. After penetration of the tissue layers, the clip is advanced as the needle is retracted. With needle retraction, the clip assumes the shape that is pre-determined thereby compressing and approximating the tissue layers. The clip may be a single solid wire of NITINOL or other highly elastic material, or it may be more complex in construction, such as being constructed of a number of wires. The invention also provides a device for clip application or deployment. In one embodiment, the needle is hollow with a taper tip, which permits it to be flush with the tip of the clip in order to decrease resistance during passage of the clip. The needle is either semi-circular, like conventional suture needles, or straight, or another curve as appropriate to the surgery being performed. The hollow needle is either attached to a hollow tube or itself continues as a hollow tube through which a number of clips can be lined serially. The hollow needle may also be partially cylindrical. This design permits a lower profile for the needle and a relatively larger clip, which utilizes the partially cylindrical needle as a “rail” on which it travels. 32=the hollow needle; 33=the tapered tip of the clip that travels inside 32; 30=a partially cylindrical hollow needle; 31=the clip that travels within 30.

FIG. 9. The delivery system could also incorporate an outer ‘stop’ over the needle. The needle would be advanced through the tissue until the tissue presses against this stop. The stop then holds the tissue in the appropriate position as mechanism advances the clip and/or retracts the needle to automatically leave the clip in the right position relative to the tissue. The clip thereby assumes a coiled or other configuration. 22=stop outside the hollow needle.

FIG. 10. An alternative construction would be for the clip described in FIG. 8 to be formed from one or more coils of one or more wires in order in order to reduce the coil diameter when the clip is deployed. For example, the clip might be formed from two concentric coils, each made from four individual strands of wire. Another alternative construction would be for the clip to be formed from a stack of thin ribbons of elastic material, again possibly held together by a polymer coating or casing. By using thin ribbons, each ribbon could again be pre-formed to reform into a much tighter coil than a single thick wire.

FIG. 11. The clip described in FIG. 8 may be designed to reform into shapes other than a circular coil or V- or U-shape. For instance, the smaller wires might reform into a floweret pattern, or another flat shape, or even a random pattern resembling a microscopic ‘Brillo’ pad, all while still resulting in a smaller final shape or profile due to the smaller diameter bends in each individual wire element. 45=floret pattern at end of clip; 46=a “BRILLO” pattern at ends of clip.

FIG. 12. Another embodiment of the device is a needle tip attached to the clip, the latter of which rides over a shaft or blunt tip needle. This needle-clip device is deployed by passing the needle portion through tissue followed by retraction of the shaft resulting in coiling (or other configuration) of the clip. 24=shaft over which needle-clip combination rides; 23=needle-clip combination.

FIG. 13. The needle-clip described in FIG. 11 may be slotted or “keyed” so that orientation of the needle is maintained. 42=square slot in needle-clip for orientation.

FIG. 14. Another embodiment of the device is a plurality of clips mounted on a ring, which can be separated or dismantled. Each individual clip is a tubular coil (with a hollow center) attached to a solid needle tip. The clips on the ring are passed through the graft vessel initially. After identifying and making an opening in the target vessel, the graft with the premounted device is brought the target vessel and the needle component is individually passed through the target vessel from the inside out, i.e., through the intima and exiting the adventitia layer. The ring with the needles is retracted, thereby deploying all the clips, which assume a coiled or other configuration. This device is applicable to the connection of any hollow viscus. 50=graft; 51=target vessel.

FIG. 15. Another embodiment of the device is a plurality of needle-clips (clip attached to the needle tip). The coil component of the each needle-clip is attached to one of the shafts on the ring. For vascular anastomosis, after passing the needle-coil combination through the graft tissue, an opening is made in the target vessel. The needle-clip is individually passed through the target vessel from the intima to adventitia. After completing passage of all needle-clip combinations, the ring along with the shafts is retracted with resultant simultaneous deployment of all needle-clips. The clip components assume a coiled or other configuration with resultant tissue approximation. This device is applicable to the connection of any hollow viscus. 50=graft; 51=target vessel.

FIG. 16. The shaft can be made in a noncylindrical configuration, e.g., with facets, so as to permit the needle-coil combination to be keyed thereby preventing twisting of the needle-coil on the shaft. Other configurations are also possible, such as a V-shape or a stop or head on the proximal end, so as to permit apposition and compression of tissues. 52=shaft that is square or faceted configuration.

FIG. 17. The ring for either the clip or needle-clip application is constructed so as to permit separation and dismantling in order to facilitate removal from the graft after construction of the anastomosis. The ring can be coupled and decoupled in one or more regions. 53=ring that is able to be coupled and decoupled

FIG. 18. Another embodiment of the needle-clip device is for the needle-clip to travel within a hollow tube. After deployment of the needle-clip by pushing it out of the hollow tube, the clip component assumes a coiled or other configuration. This needle-clip within a hollow tube and be extended to a plurality of needle-clips loaded within a series of hollow tubes mounted on a ring. Again, after tissue penetration, the ring with hollow tubes may be withdrawn with resultant clip assuming either a coiled or other configuration (e.g., everted or floret pattern) for tissue approximation.

FIG. 19. The invention also provides for a mechanism for clip removal. The clip is designed to permit simple disengagement if misdeployed. Using a pair of forceps, the clip can be removed from the tissue layers. Should this not be possible, a straightening tool will facilitate removal of the clip. The clip removal device, designed to minimize tissue injury during clip removal should the clip not be situated in the desired location, is intended to straighten the coiled wire or hollow clip. The tip of the device grasps the tip of the clip, brings it into a hollow shaft, thereby straightening the clip for easy removal. Another embodiment of the removal device is based on passage of a shaft into the coiled clip thereby straightening it for removal.

### DETAILED DESCRIPTION OF THE INVENTION

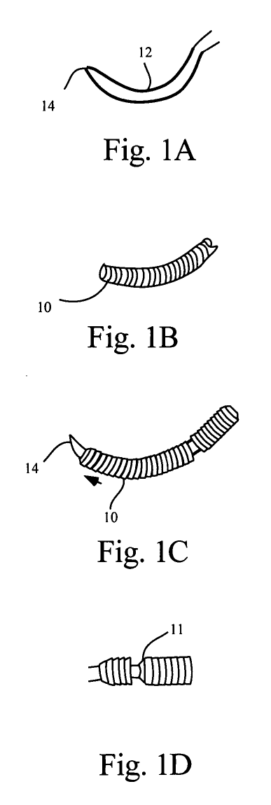

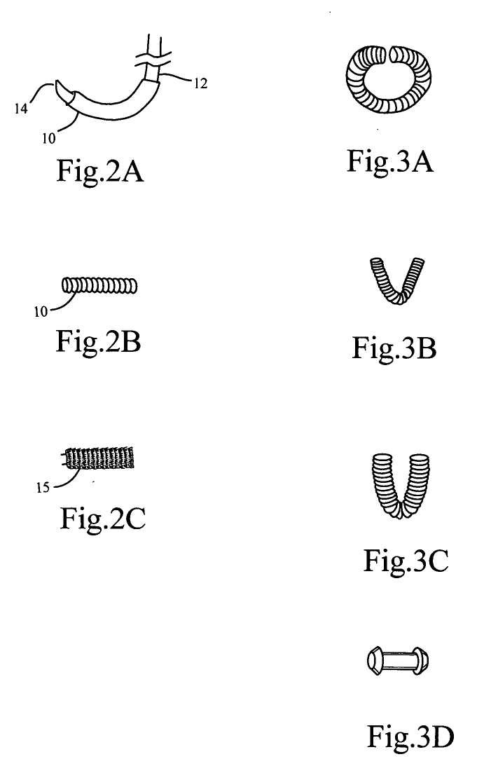

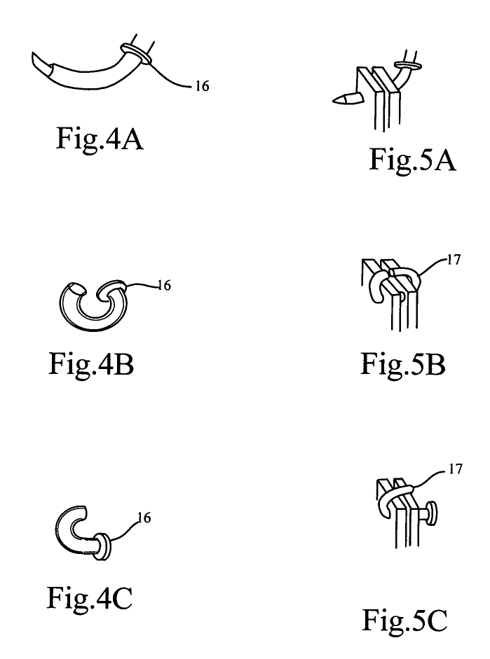

In one embodiment of the device, an elastic or highly flexible fastener or clip 10, which is in a predetermined coiled configuration, travels over shaft 12, e.g., a solid needle with a taper tip 14 (FIGS. 1A and 2A). The clip is a thin flexible coil or ‘tube’ (FIGS. 1B, 1C, 1D, 2B, and 2C). After tissue penetration the clip is advanced and the needle retracted resulting in clip deployment. The tip of the clip 10 can be slightly tapered 11 so as to be flush with the solid needle tip in order to minimize resistance when passing through tissue. After tissue penetration, the clip fastener is advanced and the shaft/needle retracted resulting in clip deployment and tissue apposition. The clip 10 may assume a variety of configurations once deployed. The clip may coil onto itself thereby approximating the tissue layers (FIG. 3A). The clip may also become V-shaped or U-shaped to approximate the tissues (FIGS. 3B and 3C). Finally, the clip may also become dumbbell in shape thereby anchoring itself on the outer layers of the tissues (3D). The coil may be wound from a single wire 10 (FIG. 2B), or it may be wound from a number of wires 15 (FIG. 2C), which would make it much stronger. The tubular clips may be formed with ‘heads’ or ‘stops’ on their proximal ends (away from the tip of the clip) 16 as shown in FIGS. 4A, 4B, and 4C. These heads might be formed from the wires, or they might be a separate element, or they might be a continuation of the polymer on the clip. In use, the delivery needle and the distal portion of the tubular clip would be passed through the tissue until the tissue pressed against the head of the clip, which would be large enough not to pass through the tissue (FIGS. 5A and 5C). While holding enough forward pressure on the clip to prevent the tissue from sliding distally, the needle would be retracted, allowing the distal portion of the clip to reform into a coiled or U-shaped configuration 17 (FIGS. 5B and 5C). The tissue would remain compressed between the proximal head and the distal coil. Thus, with the stop on the clip 16, only the distal aspect of the clip has to coil or bend to achieve tissue apposition. The head on the clip would not only simplify the surgical issues associated with positioning and delivering the clips appropriately, it would also reduce the volume of material associated with one side of the clip to the absolute minimum needed to prevent it from passing through the tissue.

This tubular clip could be made from a simple wire coil or multiple wires coiled together (FIGS. 2B and 2C). For instance, a clip made for coronary artery anastomosis might pass over a needle of approximately 0.007″ in diameter, and the coil might have an inner diameter of 0.0075″ and an outer diameter of 0.0095″. The wire coil in this example might be wound from a single wire with a diameter of 0.001″, but it might also be wound from a number of wires, for example four wires, which would make it much stronger. The clip might be made from many more wires, until the wires might run much more longitudinally, and less circumferentially, around the delivery needle. These might be bare wires, or they might again be held together with an adhesive or polymer coating of appropriate materials as described above.

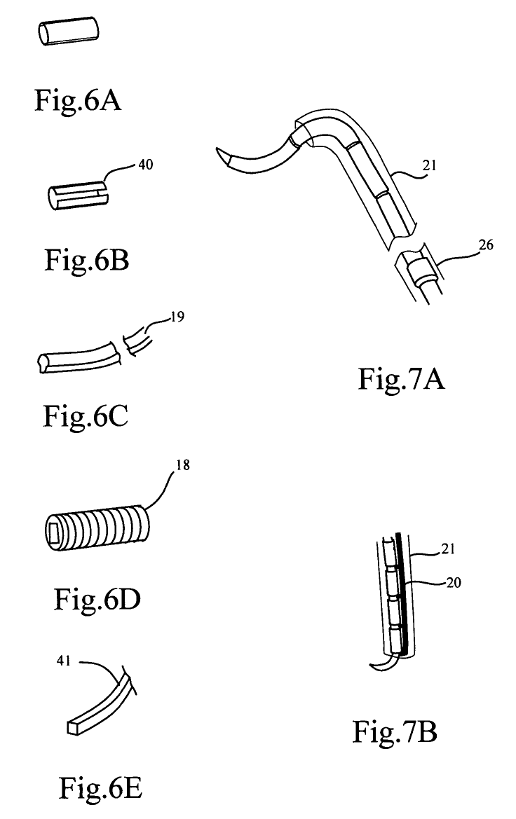

The clips may have a coiled body and a square cross-section, rather than a round cross-section. The clips may be constructed of bare wires, or they may be held together with an adhesive or polymer coating of appropriate materials as described above. The solid needle over which the clip travels may have a corresponding square cross-section 18 and 41 (FIGS. 6D and 6E), and/or a key/slot 19 and 40 (FIGS. 6B and 6C) rather than a round cross-section (FIG. 6A), in order to maintain clip orientation.

The invention also provides a device for clip application or deployment (FIGS. 7A and 7B). The main advantage of this clip design is that multiple self-closing clips can be loaded serially so as to facilitate rapid tissue approximation or vessel anastomosis by permitting rapid multiple firings (FIG. 7A). This clip is placed in series over the needle so that with each activation, the first clip is advanced and deployed, followed by a subsequent clip which is ready for deployment. As each clip is deployed, the next clip could be advanced into position near the distal tip of the needle. The needle and clips are connected to a handle with a push rod 26 or an actuating mechanism 20 which permits calibrated passage and advancement of each individual clip (FIG. 7B). There may be a housing 21 over all but the needle tip and the most distal clip in order to avoid bunching of the clips when advanced.

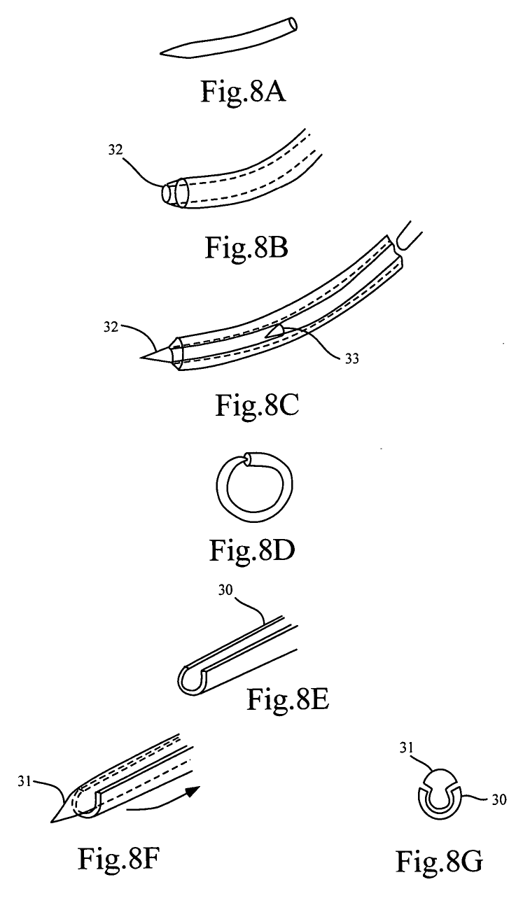

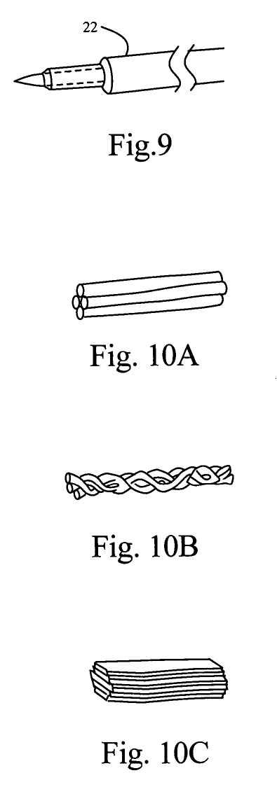

Another embodiment comprises a flexible composite clip that travels within the lumen of a complete 32 or partially cylindrical hollow needle 30 (FIGS. 8A, 8B, 8C, 8D, 8E, 8F, and 8G). The hollow needle may have a stop incorporated onto the outside of the needle 22 to facilitate clip delivery (FIG. 9). The tip of the clip 33 lies flush with the tip of the needle. After penetration of the tissue layers, the clip is advanced as the needle is retracted. With needle retraction, the clip assumes the shape that is pre-determined thereby compressing and approximating the tissue layers (FIG. 8D). With the partially cylindrical hollow needle 30, the clip 31 utilizes the needle as a “rail” and thus may be larger relative to the needle (FIGS. 8E, 8F, and 8G). While the clip may be a single solid wire of NITINOL or other highly elastic material, the wire is likely to be more complex in construction (FIGS. 10A, 10B, and 10C). It may, for instance, be made of a number of wires (FIG. 10A), for example seven wires, in order to dramatically reduce the coil diameter when the clip is deployed. Whereas a solid wire of 0.004″ would have a deployed coil diameter of approximately 0.040″, a wire made of seven wires, each with a diameter of 0.0013″, could relax into a coil with a diameter of approximately 0.013″. This would be a significant advantage for anastomosis of very small vessels. These seven smaller wires might be twisted to hold them together, both when straight and when released into a coil shape. Another, alternative construction would be to take seven wires with a diameter of 0.001″, and to surround them in a tightly wound coil of wire (or ribbon) with a diameter (or thickness) of 0.0005″, again forming a ‘wire’ with an overall diameter of 0.004″. The ‘wire’ might also have a composite construction, with a number of NITINOL or stainless steel wires held together by an adhesive or polymer. It might be advantageous for this polymer to allow the wires to slide relative to each other, to preserve their ability to relax into a smaller diameter coil. The number of smaller wires might, for instance, be held together within a thin-walled tube of a polymer such as polytetrafluoroethylene (PTFE), ePTFE, polypropylene, or another polymer.

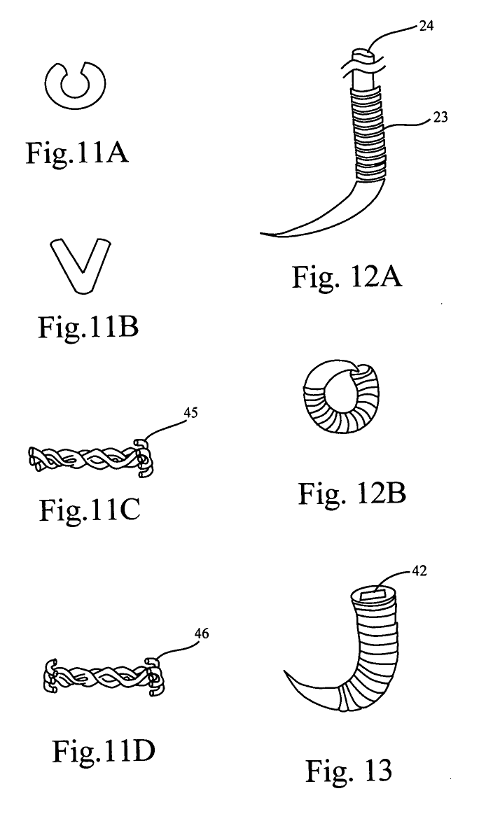

Another alternative construction would be for the clip to be formed from one or more coils of one or more wires (FIG. 10B). For example, the clip might be formed from two concentric coils, each made from four individual strands of wire. Yet another alternative construction would be for the clip to be formed from a stack of thin ribbons of elastic material, again possibly held together by a polymer coating or casing (FIG. 10C). By using thin ribbons, each ribbon could again be pre-formed to reform into a much tighter coil than a single thick wire. The clip also may be designed to reform into shapes other than a coil (FIGS. 11A, 11B, 11C, and 11D). For instance, the smaller wires might reform into a floret pattern 45, or another flat shape, or even a random pattern resembling a microscopic ‘BRILLO’ pad 46, all while still resulting in a smaller final shape or profile due to the smaller diameter bends in each individual wire element.

Another embodiment is a combination of a clip attached to a needle tip (FIG. 12A). The clip component 23 of the needle-clip rides over solid shaft 24. When the shaft is removed the clip component coils or assumes another configuration to appose tissues (FIG. 12B). The lumen of the clip component of the needle-clip and be slotted or of a square configuration 42 so as to maintain needle tip orientation during deployment (FIG. 13).

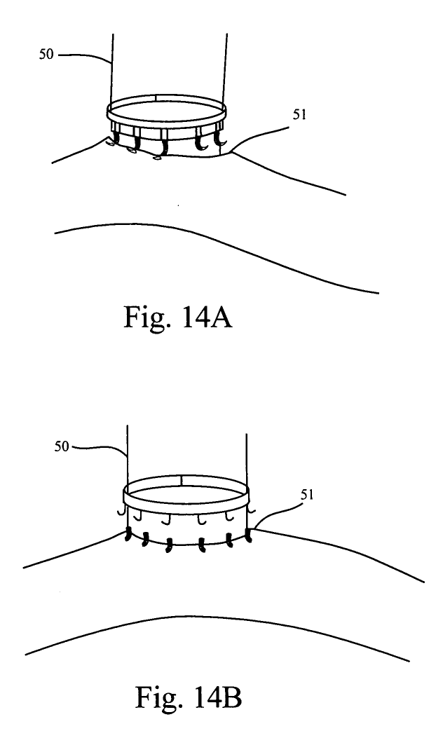

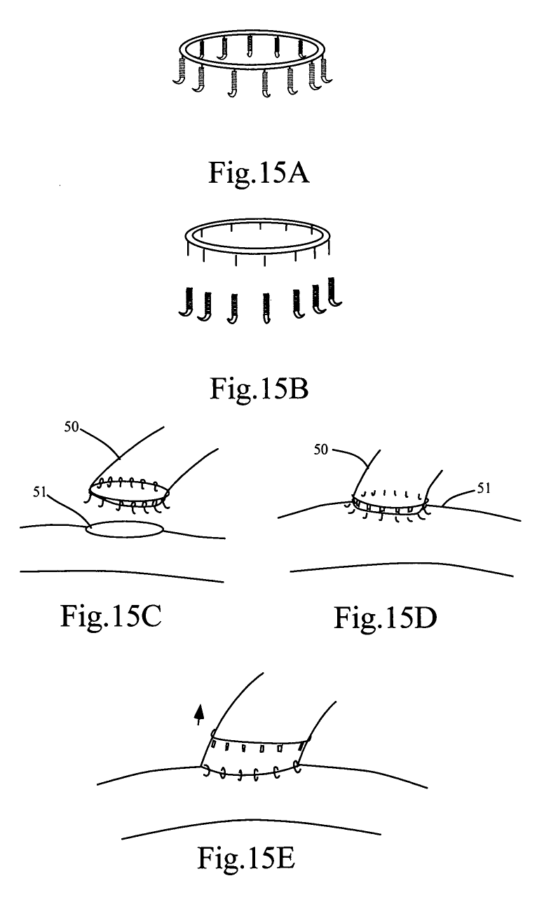

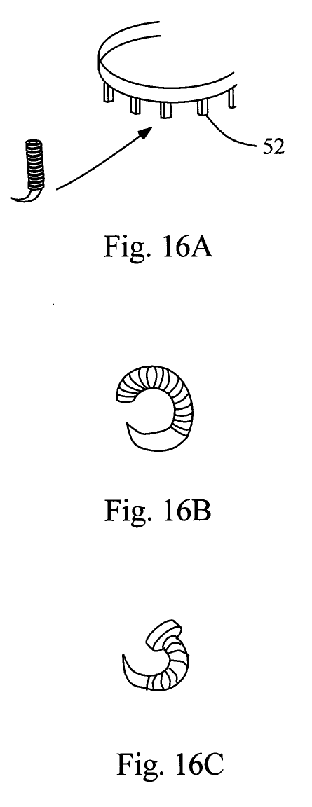

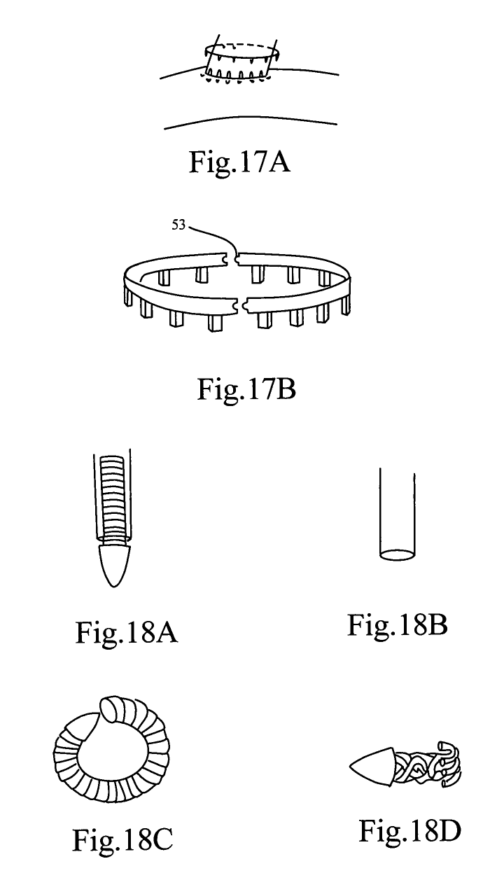

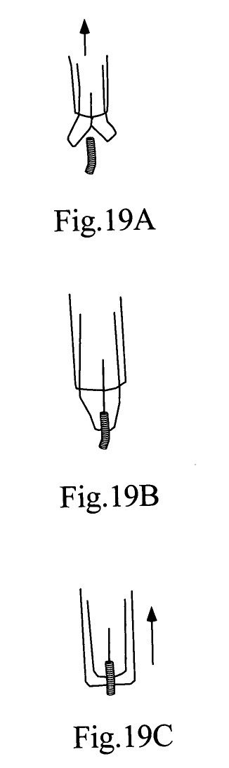

Another embodiment of the device is a plurality of clips mounted on a ring, which can be separated or dismantled (FIGS. 14, 15, 16, and 17). Each individual clip is a tubular coil (with a hollow center) attached to one of many solid needles on the ring. The clips on the ring are passed through the graft vessel 50 initially (FIG. 14A). After identifying and making an opening in the target vessel 51, the graft with the premounted device is brought the target vessel and the needle component is individually passed through the target vessel from the inside out, i.e., through the intima and exiting the adventitia layer. The ring with the needles is retracted, thereby deploying all the clips, which assume a coiled or other configuration (FIG. 14B). Thus, performing a rapid vascular anastomosis is possible in an interrupted fashion (as opposed to continuous suture). An extension of the ring concept is the use of a plurality of needle-clips (clip attached to the needle tip). Each individual clip is a coil with a hollow center attached to a solid needle tip. The coil component of the each needle-clip is attached to one of the many shafts on the ring (FIGS. 15A, 15B, 15C, 15D, and 15E). For vascular anastomosis, after passing the needle-coil combination through the graft tissue 50, an opening is made in the target vessel 51. The needle-clip is individually passed through the target vessel 51 from the intima to adventitia (FIGS. 15C and 15D). After completing passage of all needle-clip combinations, the ring along with the shafts is retracted with resultant simultaneous deployment of all needle-clips (FIG. 15E). The clip components assume a coiled or other configuration with resultant tissue approximation. Also, the shaft can be made in a noncylindrical configuration, e.g., with facets or a square configuration 52, so as to permit the needle-coil combination to be keyed thereby preventing twisting of the needle-coil on the shaft (FIGS. 16A, 16B, and 16C). The ring is constructed so as to permit separation and dismantling in order to facilitate removal from the graft after construction of the anastomosis (FIGS. 17A and 17B). The ring can be coupled and decoupled in one or more regions 53. Another embodiment of this needle-clip on a ring concept is for the clip component to be traveling within hollow tubes which are mounted in a circular fashion (FIGS. 18A, 18B, 18C, and 18D). Again, after tissue penetration, the ring with hollow tubes may be withdrawn with resultant clip assuming either a coil or dumbbell shape or floret shape (FIGS. 18C and 19D) for tissue approximation and achieving an anastomosis.

In the second embodiment, the needle is hollow with a taper tip, which permits it to be flush with the tip of the clip in order to decrease resistance during passage of the clip (FIG. 8A). The needle is either semi-circular, like conventional suture needles, or straight, or another curve as appropriate to the surgery being performed. The hollow needle is either attached to a hollow tube or itself continues as a hollow tube through which a number of clips can be lined serially. Alternatively, the delivery system could also incorporate an outer ‘stop’ 22 over the needle (FIG. 9). The needle would be advanced through the tissue until the tissue presses against this stop. The stop then holds the tissue in the appropriate position as mechanism advances the clip and/or retracts the needle to automatically leave the clip in the right position relative to the tissue. At the end of the line of clips, there is a pusher or puller system 26 which permits clip advancement during needle retraction at time of anastomosis. The pusher mechanism 20 is calibrated to permit advancement of one clip at a time (FIG. 7B). In order to make certain that the appropriate portion of the clip is deployed on each side of the tissue, actuation of the delivery system might advance approximately half of the clip out of the needle; withdrawing the needle from the tissue would cause the other half of the clip to deploy on the other side of the needle. Additionally, the deployment device with the needle is configured to permit endoscopic, thoracoscopic, laparoscopic or robotically assisted approaches by providing a low profile shaft which will enable it to be passed through a trocar into the body cavity of interest. An actuator at the proximal end of the shaft permits remote application of the clip.

The invention also provides for a mechanism for clip removal (FIGS. 19A, 19B, and 19C). The clip is designed to permit simple disengagement if misdeployed. Using a pair of forceps, the clip can be removed from the tissue layers. Should this not be possible, a straightening tool will facilitate removal of the clip. The clip removal device, designed to minimize tissue injury during clip removal should the clip not be situated in the desired location, is intended to straighten the coiled wire or hollow clip. The tip of the device grasps the tip of the clip (FIGS. 19A and 19B), brings it into a hollow shaft (FIG. 19C), thereby straightening the clip for easy removal. Another embodiment of the removal device is based on passage of a shaft into the coiled clip thereby straightening it for removal.

The above is a complete description of the preferred embodiments of the invention; however, various modifications of and alternatives to the embodiments described are possible without departing from the principles thereof. Therefore, nothing disclosed above should be taken to limit the scope of the invention, which is defined by the appended claims.

What is claimed is:

1. Apparatus for delivering a fastener into tissue, said apparatus comprising:
a delivery shaft;
a fastener slidably receivable over the exterior of the shaft, wherein the fastener has a resilient structure which conforms to the shape of the shaft when received over the shaft and assumes a tissue anchoring configuration when released from over the shaft; and
a sharpened distal tip extending from the shaft or the fastener to permit self-penetration of the assembly of the shaft and fastener through tissue.

2. Apparatus as in claim 1, wherein the shaft is curved.

3. Apparatus as in claim 1, wherein the shaft is straight.

4. Apparatus as in claim 1, further comprising a plurality of fasteners disposed in series on the shaft.

5. Apparatus as in claim 1, wherein the fastener has a tapered distal end against the shaft.

6. Apparatus as in claim 1, further comprising a pusher configured to pass over the shaft from the proximal shank and toward the distal tip in order to advance and deploy the fastener.

7. Apparatus as in claim 1, further comprising an anastomosis ring configured to circumscribe a free end of a tubular body member to be anastomosed to a target tissue site, wherein a plurality of needles and fasteners are disposed about the ring to pass through the free end and into the target tissue as the tip is advanced toward the target tissue.

8. Apparatus as in claim 1, wherein the fastener comprises a solid tubular body.

9. Apparatus as in claim 8, wherein the tubular body is slotted.

10. Apparatus as in claim 8, wherein the tubular body comprises a cylindrical coil.

11. Apparatus as in claim 1, wherein the anchoring configuration of the fastener is circular.

12. Apparatus as in claim 1, wherein the anchoring configuration of the fastener is folded.

13. Apparatus as in claim 1, wherein the anchoring configuration comprises at least one enlarged end segment.

14. Apparatus as in claim 13, wherein the end segment everts.

15. A self closing tissue fastener comprising:
an elastic body having a central passage for receiving a delivery rod; and
a tissue penetrating tip at a distal end of the elastic body, wherein the body is constrained to a delivery configuration when received over the delivery rod and deforms to assume an anchoring configuration when removed from the delivery rod.

16. A fastener as in claim 15, wherein the anchoring configuration of the fastener is circular.

17. A fastener as in claim 15, wherein the anchoring configuration of the fastener is folded.

18. A fastener as in claim 15, wherein the anchoring configuration comprises at least one enlarged end segment.

19. A method for fastening tissue segments, said method comprising:
advancing a fastener through said segments while the fastener is deformed over a shaft in a delivery configuration; and
releasing the fastener from the shaft so that the fastener assumes an anchoring configuration to fasten the tissue segments together.

20. Apparatus for delivering a fastener into tissue, said apparatus comprising:
a hollow needle;
a fastener that slides within the lumen of the needle, wherein the fastener has a resilient structure which conforms to the shape of the lumen and assumes a tissue anchoring configuration when released from within the hollow needle; and a sharpened distal tip extending from the needle or the fastener to permit self-penetration of the assembly of the needle and fastener through tissue.

21. Apparatus as in claim 20, wherein the hollow needle is partially cylindrical.

22. Apparatus as in claim 20, wherein the hollow needle is curved.

23. Apparatus as in claim 20, wherein the hollow needle is straight.

24. Apparatus as in claim 20, further comprising a plurality of fasteners disposed in series in the hollow needle.

25. Apparatus as in claim 20, wherein the fastener comprises at least three elastic filaments.

26. Apparatus as in claim 25, wherein the filaments are twisted about a common center axis.

27. Apparatus as in claim 25, wherein the filaments are solid packed.

28. Apparatus as in claim 20, wherein the anchoring configuration of the fastener is circular.

29. Apparatus as in claim 20, wherein the anchoring configuration of the fastener comprises at least one end segment that everts.

30. Apparatus as in claim 20, further comprising a pusher configured to pass within the lumen of the needle from the proximal shank and toward the distal tip in order to advance and deploy the fastener.
